(19) United States
(12) Patent Application Publication
(10) Pub. No.: US 2024/0016615 A1
(43) Pub. Date: Jan. 18, 2024

(54) JIG FOR POST-PROCESSING OF CUSTOMIZED ORTHOPEDIC IMPLANT AND METHOD FOR MANUFACTURING SAME

(71) Applicant: INTER-MEDI CO., LTD., Gwangju (KR)

(72) Inventors: Hyoung Gyun LIM, Gwangju (KR); Seon Shin CHO, Gwangju (KR); Ji Yeong JANG, Gwangju (KR); Su Bin KIM, Gwangju (KR)

(21) Appl. No.: 18/217,705

(22) Filed: Jul. 3, 2023

(30) Foreign Application Priority Data
Jul. 15, 2022 (KR) 10-2022-0087634

(51) Int. Cl. A61F 2/30; B33Y 40/20; B33Y 80/00; A61F 2/32

(52) U.S. Cl. CPC A61F 2/30942; B33Y 40/20; B33Y 80/00; A61F 2002/30957; A61F 2/32; A61F 2002/30985

(57) ABSTRACT

A jig is for a post-processing of a customized orthopedic implant to securely fix a customized metal implant for artificial hip joint surgery without any surface damage for post-processing of the customized metal implant after manufacturing the customized metal implant by a 3D printer, and a method for manufacturing the jig. The jig may include inner jigs 3D printed into a shape for compressing and fixing the customized metal implant by using a polymer resin material, and outer jigs made of metal for pressing and supporting the inner jigs in directions of compressing the customized metal implant, and the customized metal implant manufactured by the 3D printer according to the shape and damage degree of a patient's hip joint is securely fixed to the jig without any surface damage so that the post-processing of the metal implant can be easily performed.

### CROSS REFERENCE TO RELATED APPLICATION AND CLAIM OF PRIORITY

The present application claims the benefit under 35 USC § 119 of Korean Patent Application No. 10-2022-0087634, filed Jul. 15, 2022, the entire disclosure of which is incorporated herein by reference for all purposes.

### BACKGROUND

### 1. Field of the Invention

The present disclosure relates generally to a jig for a post-processing of a customized orthopedic implant and a method for manufacturing the same. More particularly, the present disclosure relates to a jig for a post-processing of a customized orthopedic implant by which a customized metal implant for artificial hip joint surgery can be securely fixed without any surface damage for post-processing of the customized metal implant after manufacturing the customized metal implant by using a 3D printer, and a method for manufacturing the jig.

### 2. Description of the Related Art

In general, a hip joint refers to a joint connecting a pelvis and a femur and the hip joint includes the articulation of the joint between the acetabulum and femoral head of the pelvis.

When degenerative arthritis with inflammatory lesions, avascular necrosis, and rheumatoid arthritis, etc. occur in the hip joint, a patient feels pain in the hip joint and cannot walk properly, requiring surgery. For this purpose, artificial hip joint surgery is being performed.

In the method of the existing artificial hip joint surgery, artificial joint replacement using bone cement and without using bone cement has been used, but an artificial hip joint moves in a patient who has been using the artificial hip joint for more than years after surgery, resulting in severe bone resorption around the bone cement, and thus there are cases in which revision artificial hip replacement surgery is performed. In addition, in a case in which bone loss is severe although the artificial hip replacement surgery has been performed, the artificial hip replacement surgery may be performed again because of implant loosening or instability, or periprosthetic infection.

In order to solve these problems, in consideration that each patient has the different shape and different degree of the bone damage, a customized metal implant for artificial hip joint surgery is being manufactured by using a 3D printer.

For reference, as illustrated in FIG. 1A, an artificial hip joint includes an acetabular cup 1, which is a socket connected to the surgical site of the hip joint, a liner 2 attached to the inner surface of the acetabular cup 1, a head 3 movably inserted into the liner 2, and a stem 4 extending from the head 3 and fastening to the femur, which constitute one set.

As illustrated in FIGS. 2B and 2C, the customized metal implant for artificial hip joint surgery, which is manufactured by using a 3D printer according to the shape and damage of a patient's hip joint, is connected to the surgical site of the hip joint before the acetabular cup 1 is connected thereto, and serves as a support layer on which the acetabular cup 1 is mounted.

Referring to FIGS. 2B and 2C, when the customized metal implant 100 is manufactured by using a 3D printer, the customized metal implant 100 is manufactured in a complex shape to suit the shape and damage degree of a patient's hip joint, and is manufactured to have an acetabular cup seating recessed part 101 with a predetermined depth formed in a first surface part of the customized metal implant 100 and to have a porous mesh part 102 formed on a peripheral part and a second surface part (a part in close contact with a surgical part of the hip joint) of the customized metal implant 100 to be fused with the surface of the hip joint.

In addition, when the customized metal implant 100 is manufactured by using a 3D printer, the customized metal implant 100 is finally printed together with a support (not shown) that supports a 3D printed product. This support is integrally molded at the entrance of the seating recessed part 101 and is removed after 3D printing is finished.

Accordingly, post-processing, such as surface cutting, for the entrance and surrounding part (a part from which the support was removed) of the seating recessed part 101 of the customized metal implant 100 is required to be performed.

To this end, in the past, for the post-processing of the customized metal implant 100, a jig for securely fixing the customized metal implant was manufactured through separate metal processing.

However, since the customized metal implant is manufactured in a complex shape according to the shape and damage degree of a patient's hip joint, it is difficult to manufacture the jig into a shape for fixing the customized metal implant having a complex shape.

In addition, even if a jig for fixing the customized metal implant is manufactured through metal processing, it takes a lot of cost and time to manufacture the jig, and it is difficult to modify the shape of the jig after the manufacturing, so the jig cannot be reused.

In other words, since the customized metal implant is manufactured according to the shape and damage degree of the hip joint of a specific patient, a jig to fix the metal implant is required to be newly manufactured for each customized metal implant, resulting in problems such as excessive manufacturing cost and time.

In addition, when performing the post-processing of the customized metal implant after fixing the customized metal implant to an existing metal jig, an external force is applied to a contact surface between the existing metal jig and the customized metal implant, resulting in damage to the porous mesh part of the customized metal implant.

### SUMMARY

Accordingly, the present disclosure has been made keeping in mind the above problems occurring in the related art, and the present disclosure is intended for the purpose of making jig for a post-processing of a customized orthopedic implant and a method for manufacturing the same, wherein the jig includes inner jigs 3D printed into a shape for compressing and fixing a customized metal implant by using a polymer resin material, and outer jigs made of metal for pressing and supporting the inner jigs in directions of compressing the customized metal implant, and the customized metal implant manufactured by a 3D printer according to the shape and damage degree of a patient's hip joint is securely fixed to the jig without any surface damage so that the post-processing of the customized metal implant can be easily performed.

In order to achieve the above objectives, according to one embodiment of the present disclosure, there is provided a jig for a post-processing of a customized orthopedic implant, the jig including: a first inner jig and a second inner jig made of a polymer resin material by a 3D printer, with a recessed part for seating and clamping a customized metal implant manufactured by a 3D printer being formed in an upper surface part of each of the first inner jig and the second inner jig; a first outer jig and a second outer jig manufactured of a metal material to compress the first and second inner jigs in directions for fixing the customized metal implant, with an insertion recess into which the first and second inner jigs are inserted in close contact with each other being formed in an inner side part of each of the first outer jig and the second outer jig; and a driving device coupled to the first outer jig and the second outer jig and configured to move the first outer jig or the second outer jig in a direction for compressing the first and second inner jigs, wherein while the customized metal implant seated in the recessed part is clamped by compression force of the first and second outer jigs compressing the first and second inner jigs, a post-processing of the customized metal implant is capable of being performed.

Preferably, a slide groove may be formed in an inner wall surface of each of the first and second outer jigs, and a slide protrusion inserted into and fastened to the slide groove may be formed on each of the first and second inner jigs so that the first and second inner jigs are prevented from departing upward from the first and second outer jigs.

The driving device may include: a support block to which a screw is fastened to be rotatable in place; a fixed block formed integrally on a first upper surface part of the support block and fastened to the first outer jig; a moving block disposed movably on a second upper surface part of the support block and fastened to the second outer jig; a nut block formed on a lower end part of the moving block by protruding therefrom, with the screw being rotatably inserted into and fastened to the nut block; and a rotating handle mounted on an outer part of the support block and configured to apply rotational force to the screw.

In addition, a guide rail may be formed on an upper surface part of the support block by protruding therefrom, and a guide groove into which the guide rail is inserted to be fastened thereto may be formed in a lower surface part of each of the moving block and the second outer jig.

In order to achieve the above objectives, according to another embodiment of the present disclosure, there is provided a method for manufacturing the jig for a post-processing of a customized orthopedic implant, the method including: manufacturing the customized metal implant for artificial hip joint surgery by using a 3D printer according to a shape and a damage degree of a patient's hip joint; manufacturing the first inner jig and the second inner jig made of a polymer resin material by a 3D printer for fixing the customized metal implant for post-processing thereof in such a manner that the recessed part for seating and clamping the customized metal implant is formed in each of the first inner jig and the second inner jig; and manufacturing the first outer jig and the second outer jig made of a metal material to compress the first and second inner jigs in directions for fixing the customized metal implant in such a manner that the insertion recess into which the first and second inner jigs are inserted in close contact with each other is formed in an inner side part of each of the first outer jig and the second outer jig, wherein while the customized metal implant seated in the recessed part is clamped by compression force of the first and second outer jigs compressing the first and second inner jigs, the post-processing of the customized metal implant is capable of being performed.

Preferably, the first inner jig and the second inner jig may be in direct contact with the customized metal implant and are 3D printed with a polyamide material so that the first inner jig and the second inner jig are capable of protecting a surface of the customized metal implant.

Through the solution described above, the present disclosure provides the following effects.

First, the customized metal implant manufactured by a 3D printer according to the shape and damage degree of a patient's hip joint is seated in the inner jigs 3D printed by using a polymer resin material, and the inner jigs are compressed by compression force of the outer jigs so as to securely fix the customized metal implant thereto, thereby enabling the post-processing of the customized metal implant to be easily performed without any surface damage.

Second, with the customized metal implant seated and clamped on the inner jigs made of a polymer resin material, even if external force due to post-processing such as surface cutting is transmitted to the customized metal implant, the inner jigs buffer the external force and protect the surface of the customized metal implant, thereby enabling the post-processing of the customized metal implant to be easily performed without any surface damage, and preventing a porous mesh part of the customized metal implant from being damaged.

Third, compared to existing metal jigs for fixing the customized metal implant, only the inner jigs are required to be manufactured of a polymer resin material by a 3D printer, thereby reducing manufacturing cost and time.

Fourth, the inner jigs for fixing the customized metal implant can be attached to and detached from the outer jigs, thereby enabling only the inner jigs to be replaced according to the shape of the customized metal implant, and the outer jigs to be reused.

Fifth, the jig of the present disclosure can have differentiated actions and effects increased through various embodiments.

### BRIEF DESCRIPTION OF THE DRAWINGS

The above and other objectives, features, and other advantages of the present disclosure will be more clearly understood from the following detailed description when taken in conjunction with the accompanying drawings, in which:

FIG. 1A is a schematic diagram illustrating the configuration of an artificial hip joint set;

FIG. 1B is a diagram illustrating a case of an actual patient who needs a customized implant;

FIG. 2A is a diagram illustrating the state of use of the customized implant;

FIGS. 2B and 2C are schematic diagrams illustrating an example of the appearance of a customized metal implant for artificial hip joint surgery;

FIG. 3 is a conceptual diagram illustrating a jig for a post-processing of a customized orthopedic implant according to the present disclosure;

FIG. 4 is an exploded perspective view illustrating the jig for a post-processing of a customized orthopedic implant according to a first embodiment of the present disclosure;

FIG. 5 is an assembly perspective view illustrating the jig for a post-processing of a customized orthopedic implant according to the first embodiment of the present disclosure;

FIG. 6 is an exploded perspective view illustrating a jig for a post-processing of a customized orthopedic implant according to a second embodiment of the present disclosure; and

FIG. 7 is an assembly perspective view illustrating the jig for a post-processing of a customized orthopedic implant according to the second embodiment of the present disclosure.

FIG. 8 is an actual X-ray image of a patient who underwent surgery by inserting a patient-specific implant.

### DETAILED DESCRIPTION

Hereinafter, exemplary embodiments of the present disclosure will be described in detail with reference to the accompanying drawings.

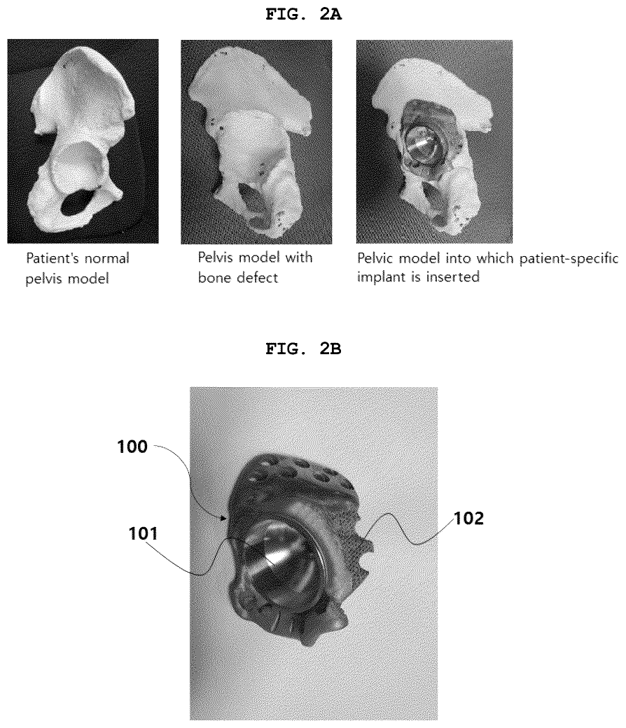

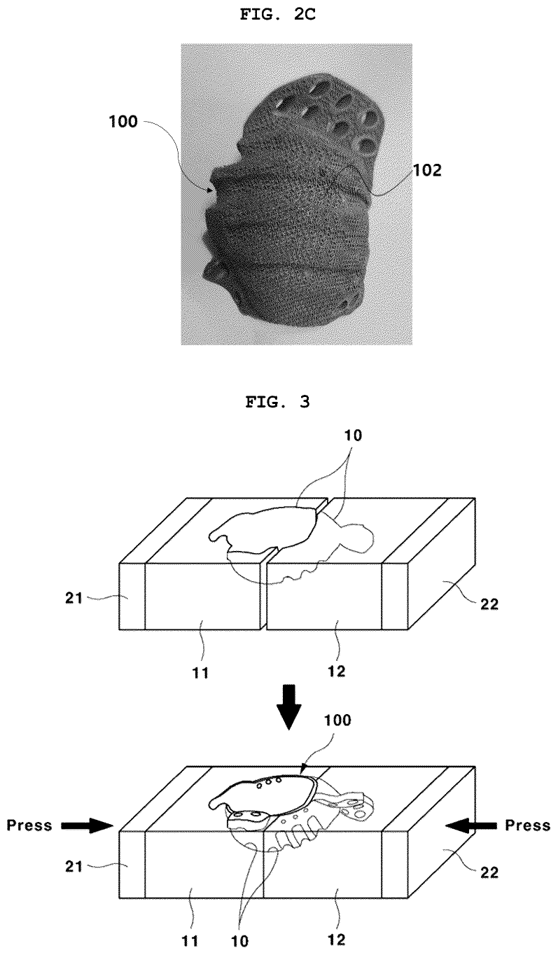

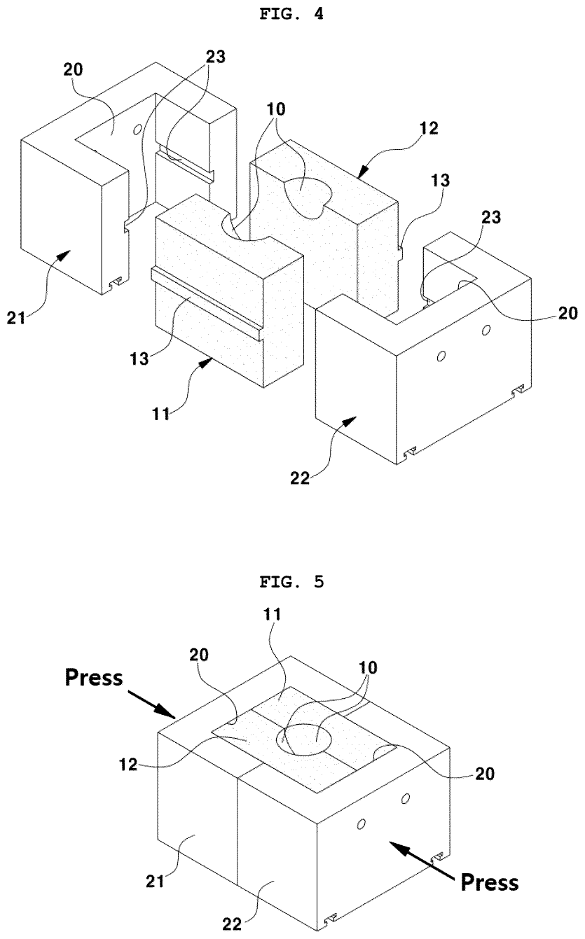

FIG. 3 is a conceptual diagram illustrating a jig for a post-processing of a customized orthopedic implant according to the present disclosure, FIG. 4 is an exploded perspective view illustrating the jig for a post-processing of a customized orthopedic implant according to a first embodiment of the present disclosure, and FIG. 5 is an assembly perspective view illustrating the jig for a post-processing of a customized orthopedic implant according to the first embodiment of the present disclosure.

As illustrated in FIGS. 3 to 5, the jig according to the present disclosure includes the first inner jig 11 and the second inner jig 12 3D printed with a polymer resin material for the seating and clamping of the customized metal implant 100 manufactured by a 3D printer, and the first outer jig 21 and the second outer jig 22 made of a metal material to compress the first and second inner jigs 11, 12 in the direction of fixing the customized metal implant 100.

The customized metal implant 100 is used to perform conservative treatment by replacing a patient's bone defective part or by being connected to a damaged joint, and may be manufactured by using a 3D printer within an approved range based on the patient's image information according to the size and shape of the patient's defective part.

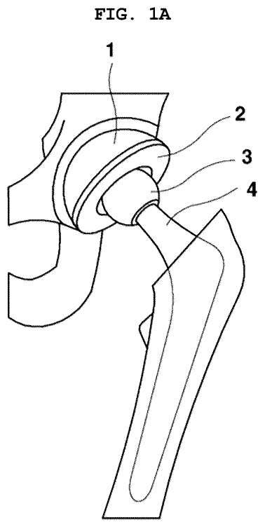

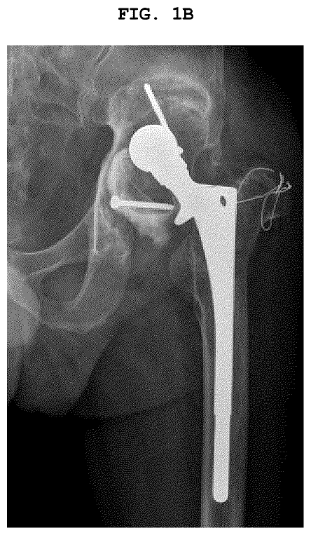

In other words, as illustrated in FIGS. 2B and 2C, the customized metal implant, which is manufactured by using a 3D printer according to the shape and damage degree of a patient's hip joint, is connected to the surgical site of the hip joint before the acetabular cup 1 illustrated in FIG. 1A is connected thereto and serves as a support layer on which the acetabular cup 1 is mounted.

To this end, when the customized metal implant 100 is manufactured by using a 3D printer, the customized metal implant 100 is manufactured in a complex shape according to the shape and damage degree of a patient's hip joint, and is manufactured to have the acetabular cup seating recessed part 101 with a predetermined depth formed in a first surface part of the customized metal implant 100 as illustrated in FIG. 2B and to have a porous mesh part 102 formed on a peripheral part and a second surface part thereof (a part in close contact with a surgical part of the hip joint) to be fused with the surface of the hip joint as illustrated in FIG. 2C.

In this case, when the customized metal implant 100 is manufactured by using a 3D printer, the customized metal implant 100 is finally printed with a support (not shown) supporting the customized metal implant, which is a 3D printed product. This support is integrally molded at the entrance of the seating recessed part 101 and then is removed after 3D printing is finished.

Accordingly, in order to apply the customized metal implant 100 to an actual surgical site, post-processing, such as surface cutting, for the entrance and surrounding part (a part from which the support is removed) of the seating recessed part 101 is required to be performed, and for the post-processing, the customized The metal implant 100 is required to be clamped without any surface damage.

To this end, the first inner jig 11 and the second inner jig 12 are made of a polymer resin material by a 3D printer so that the customized metal implant 100 can be clamped without any surface damage for post-processing, and a recessed part 10 for seating and clamping the customized metal implant 100 manufactured by a 3D printer is formed in the upper surface part of each of the first inner jig 11 and the second inner jig 12.

That is, during 3D printing, the recessed part 10 of each of the first inner jig 11 and the second inner jig 12 is formed in a shape corresponding to the shape of the peripheral part and lower surface part of the customized metal implant 100 so that the peripheral part and lower surface part of the customized metal implant 100 can be in close contact with and clamped in the recessed part 10.

Preferably, the first inner jig 11 and the second inner jig 12 are in direct contact with the customized metal implant 100 and are 3D printed with a polyamide material so that the first inner jig 11 and the second inner jig 12 can buffer external force transmitted to the customized metal implant 100 and protect the surface of the customized metal implant 100.

In addition, the first outer jig 21 and the second outer jig 22 are manufactured of a metal material to compress the first and second inner jigs 11 and 12 in directions for fixing the customized metal implant 100 and are manufactured such that an insertion recess 20 into which the first and second inner jigs 11, 12 are inserted in close contact with each other is formed in an inner side part of each of the first outer jig 21 and the second outer jig 22.

Accordingly, after connecting the first and second outer jigs 21, 22 to the external fastening means (e.g., a chuck), and bringing the first and second inner jigs 11, 12 into close contact with each other to seat the customized metal implant 100 in the recessed part 10 of the first and second inner jigs 11, 12, the first and second outer jigs 21 and 22 are respectively moved in the directions of compressing the first and second inner jigs 11 and 12 by the external fastening means.

Next, when the first and second outer jigs 21, 22 are moved in the directions of compressing the first and second inner jigs 11, 12, the first and second inner jigs 11, 12 are inserted into the insertion recesses 20 of the first and second outer jigs 21, 22 to share the insertion recesses and are compressed by the clamping force of the first and second outer jigs 21, 22.

In this case, when the first and second inner jigs 11 and 12 are compressed, the peripheral part of the customized metal implant 100 seated in the recessed part 10 of the first and second inner jigs 11 and 12 is clamped and fixed.

Accordingly, in the state in which the customized metal implant 100 is easily clamped in the recessed part 10 of the first and second inner jigs 11, 12 by the compression force of the first and second outer jigs 21, 22, post-processing, such as surface cutting, for the entrance and surrounding part (a part from which the support is removed) of the seating recessed part 101 of the customized metal implant 100 may be performed precisely.

Preferably, in order to prevent the first and second inner jigs 11 and 12 from departing from the first and second outer jigs 21 and 22, a slide groove 23 is formed in the inner wall surface of each of the first and second outer jigs 21, 22, and a slide protrusion 13 inserted into and fastened to the slide groove 23 is formed on each of the first and second inner jigs 11, 12.

Accordingly, when the first and second inner jigs 11 and 12 are compressed by the first and second outer jigs 21 and 22, the first and second inner jigs 11 and 12 can be easily prevented from escaping upward by the locking force of the slide protrusion 13 inserted into the slide groove 23.

Accordingly, with the customized metal implant 100 seated and clamped on the first and second inner jigs 11, 12 made of a polymer resin material, even if the external force of post-processing such as surface cutting is transmitted to the customized metal implant 100, the first and second inner jigs 11, 12 buffer the external force and protect the lower surface part and the peripheral part of the customized metal implant 100, so the post-processing of the customized metal implant 100 can be easily performed without any surface damage, and accordingly, the porous mesh part 102 of the customized metal implant 100 can be easily prevented from being damaged.

Meanwhile, the jig according to the present disclosure further includes a driving device 30 coupled to the first outer jig 21 and the second outer jig 22 and configured to move the first outer jig 21 or the second outer jig 22 in a direction for compressing the first and second inner jigs 11, 12.

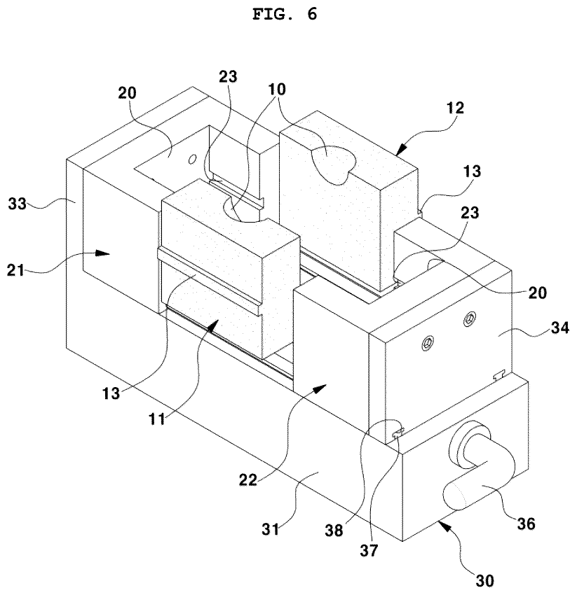

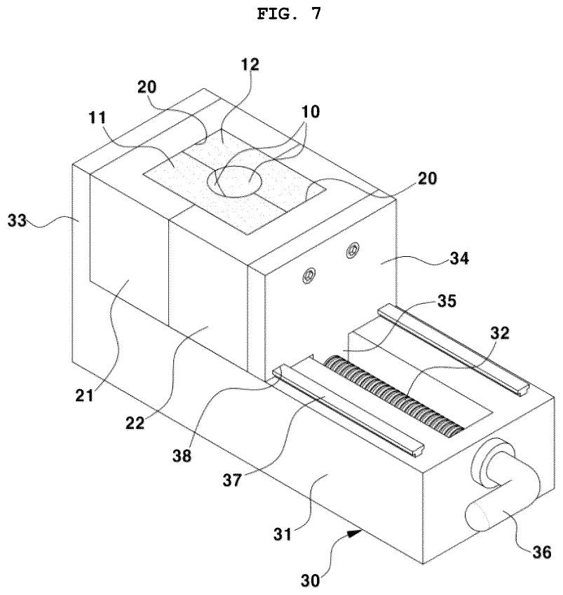

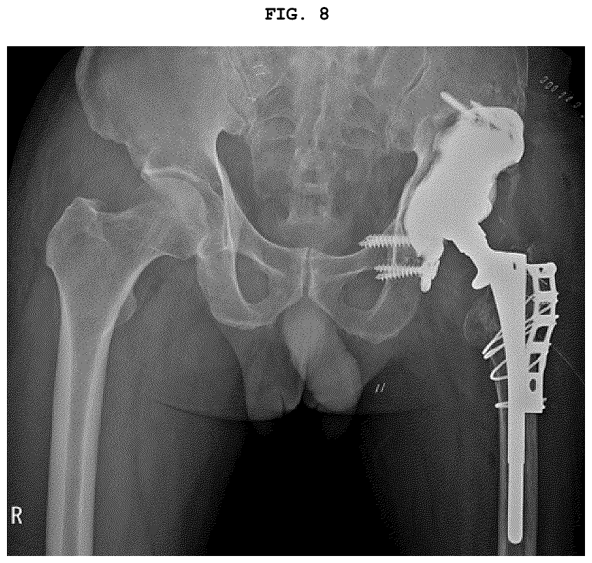

To this end, as illustrated in FIGS. 6 and 7, the driving device 30 includes a support block 31, with a screw 32 being fastened to an open upper surface part of the support block 31 so that the screw 32 is rotatable in place, a fixed block 33 formed integrally on a first upper surface part of the support block 31 and fastened to the first outer jig 21, and a moving block 34 disposed movably on a second upper surface part of the support block 31 and fastened to the second outer jig 22.

Particularly, a nut block 35 is formed integrally on a lower end part of the moving block 34 by protruding therefrom, with the screw 32 being rotatably inserted into and fastened to the nut block 35.

In addition, a guide rail 37 with a “T”-shaped cross section is formed on an upper surface part of the support block 31 by protruding therefrom so as to ensure the rectilinear movement of the moving block 34, and a guide groove 38 into which the guide rail 37 is inserted to be fastened thereto is formed in the lower surface part of each of the moving block 34 and the second outer jig 22.

Preferably, a rotating handle 36 that is connected to the screw 32 and applies rotational force to the screw 32 is mounted on an outer part of the support block 31, or a motor may be connected to the screw 32 for an electric method.

Here, a method for manufacturing the jig of the present disclosure having the components described above and a method for using the jig are described as follows.

First, the customized metal implant 100 for artificial hip joint surgery is manufactured by using a 3D printer according to the shape and damage degree of a patient's hip joint.

For example, as described above, when the customized metal implant 100 is manufactured by using a 3D printer, the customized metal implant 100 is manufactured in a complex shape according to the shape and damage degree of a patient's hip joint. The customized metal implant 100 may be manufactured to have the acetabular cup seating recessed part 101 with a predetermined depth formed in the first surface part as illustrated in FIG. 2B, and to have the porous mesh part 102 formed on the peripheral part and the second surface part (the part in close contact with a surgical part of the hip joint) to be fused with the surface of the hip joint as illustrated in FIG. 2C.

Next, the first inner jig 11 and the second inner jig 12 for fixing the customized metal implant 100 for the post-processing of the customized metal implant 100 are manufactured of a polymer resin material by a 3D printer and are manufactured in such a manner that the recessed part 10 for seating and clamping the customized metal implant 100 is formed in each of the first inner jig 11 and the second inner jig 12.

As described above, the first inner jig 11 and the second inner jig 12 are manufactured of a polymer resin material (e.g., polyamide) by a 3D printer so that the customized metal implant 100 can be clamped without any surface damage for post-processing and are manufactured in such a manner that the recessed part 10 for seating and clamping the customized metal implant 100 manufactured by a 3D printer is formed in the upper surface part of each of the first inner jig 11 and the second inner jig 12.

Particularly, during 3D printing, the recessed part 10 of each of the first inner jig 11 and the second inner jig 12 is formed in a shape corresponding to the shape of the peripheral part and the lower surface part of the customized metal implant 100 so that the peripheral part and the lower surface part of the customized metal implant 100 can be in close contact with and clamped in the recessed part 10.

Next, the first outer jig 21 and the second outer jig 22 are manufactured of a metal material to compress the first and second inner jigs 11, 12 in directions for fixing the customized metal implant 100 first and second inner jigs 11, 12, and are manufactured such that the insertion recess 20 into which the first and second inner jigs 11, 12 are inserted in close contact with each other is formed in the inner side part of each of the first outer jig 21 and the second outer jig 22.

In this case, the first and second outer jigs 21, 22 may be connected to the external fastening means (e.g., a chuck), but the first outer jig 21 may be fixedly fastened to the fixed block 33 of the driving device 30, and the second outer jig 22 may be movably fastened to the moving block 34 of the driving device 30.

Accordingly, when the rotating handle 36 located outside the support block 31 is turned or a motor (not shown) is driven, the screw 32 rotates in place.

In this case, the screw 32 is inserted into and fastened to the nut block 35 formed on the lower end part of the moving block 34, and the guide rail 37 of the support block 31 is inserted into and fastened to the guide groove 38 of the moving block 34, and thus when the screw 32 rotates in place, the moving block 34 may be easily moved in a straight line.

In addition, with the first outer jig 21 fixed to the fixed block 33, the second outer jig 22 fastened to the moving block 34 may move rectilinearly together with the moving block 34.

Accordingly, after bringing the first and second inner jigs 11, 12 into close contact with each other and seating the customized metal implant 100 in the recessed part 10 of the first and second inner jigs 11, 12, the second outer jig 22, together with the moving block 34, may be moved in the direction of compressing the first and second inner jigs 11 and 12.

Next, when with the first outer jig 21 fixed, the second outer jig 22 is moved in the direction of compressing the first and second inner jigs 11, 12, the first and second inner jigs 11, 12 are inserted into the insertion recesses 20 of the first and second outer jigs 21, 22 to share the insertion recesses and are compressed by the clamping force of the first and second outer jigs 21, 22.

In this case, when the first and second inner jigs 11, 12 are compressed, the peripheral part of the customized metal implant 100 seated in the recessed part 10 of the first and second inner jigs 11, 12 is clamped and fixed therein.

Accordingly, while the customized metal implant 100 is easily clamped in the recessed part 10 of the first and second inner jigs 11, 12 by compression force of the first and second outer jigs 21, 22, post-processing, such as surface cutting, for the entrance and surrounding part (a part from which the support is removed) of the seating recessed part 101 of the customized metal implant 100 may be performed precisely.

Accordingly, with the customized metal implant 100 is seated and clamped on the first and second inner jigs 11, 12 made of polyamide, even if external force due to post-processing such as surface cutting is transmitted to the customized metal implant 100, the first and second inner jigs 11, 12 buffer the external force and protect the lower surface part and the peripheral part of the customized metal implant 100, so the post-processing of the customized metal implant 100 can be easily performed without any surface damage, and accordingly, the porous mesh part 102 of the customized metal implant 100 can be easily prevented from being damaged.

In addition, only the first and second inner jigs 11, 12 for fixing the customized metal implant (100) may be attached to and detached from the first and second outer jigs 21, 22, and thus according to the shape of the customized metal implant 100, only the first and second inner jigs 11, 12 may be replaced, and the outer jigs may be reused.

Meanwhile, a coating layer may be formed in the insertion recess 20 each of the first outer jig 21 and the second outer jig 22 so as to improve weather resistance and wear resistance of a metal surface.

The coating material of such a coating layer may include triglycidyl p-aminophenol 25% by weight, alkoxyalkyl monoamine 18% by weight, hafnium 12% by weight, organic acid magnesium 16% by weight, titanium oxide (TiO2) 9% by weight, aluminum oxide (AIO2) 10% by weight, and Transcutol 10% by weight, and the coating thickness of the coating layer may be 9 μm.

Triglycidyl p-aminophenol and alkoxyalkyl monoamine serve to prevent corrosion, weather resistance, and discoloration, etc., and hafnium, which is a transition metal element with wear resistance and weather resistance, has excellent waterproof and corrosion resistance.

Organic acid magnesium serves to impart alkali resistance and wettability to the surface of a coating film, Transcutol serves as a surfactant surface, and titanium oxide and aluminum oxide are added for the purpose of fire resistance and chemical stability.

A reason in which the ratio of the components and the coating thickness are limited to the numerical values as described above is that the ratio shows optimal weather resistance and abrasion resistance improvement effect as a result of a present inventor's analysis through test results through several failures.

In addition, a stain-resistant coating layer made of a stain-resistant coating composition may be applied to the slide groove 23 of the first outer jig 21 and the second outer jig 22 to improve stain resistance.

The stain-resistant coating composition includes teradeoxycholate and esterquat in a molar ratio of 1:0.01 to 1:2, and the total content of teradeoxycholate and esterquat is 1 to 10% by weight based on a total aqueous solution.

The molar ratio of teradeoxycholate and esterquat is preferably 1:0.01 to 1:2. When the molar ratio is out of the above range, the applicability of teradeoxycholate and esterquat to the slide groove 23 is reduced or water adsorption on the surface of the slide groove increases after the application of teradeoxycholate and esterquat thereto, thereby removing the coating film.

The teradeoxycholate and esterquat are preferably 1 to 10% by weight of the total aqueous solution of the composition. When the ratio is less than 1% by weight, the coating property of the slide groove 23 is deteriorated, and when the ratio exceeds 10% by weight, crystallization is likely to occur due to the increase of the thickness of the coating film.

Meanwhile, as a method of applying the stain-resistant coating composition to the slide groove 23, a spraying method is preferable. In addition, the thickness of the final coating film of the slide groove 23 is preferably 800 to 2400 Å, and more preferably is 900 to 2000 Å. When the thickness of the coating film is less than 800 Å, there is a problem of deterioration in the case of high-temperature heat treatment, and when the thickness exceeds 2400 Å, there is a disadvantage in that crystallization of a coated surface is likely to occur.

In addition, the stain-resistant coating composition may be prepared in such a manner that 0.1 mole of teradeoxycholate and mole of esterquat are added to 1000 ml of distilled water and then stirred.

The reason why the ratio of the components and the thickness of the coating film are limited to the numerical values as described above is that the ratio shows an optimal stain-resistant coating effect as a result of the present inventor analyzing test results through several failures.

What is claimed is:

1. A jig for a post-processing of a customized orthopedic implant, the jig comprising:
a first inner jig and a second inner jig which are made of a polymer resin material by a 3D printer, with a recessed part for seating and clamping a customized metal implant manufactured by a three-dimensional (3D) printer formed in an upper surface part of each of the first inner jig and the second inner jig;
a first outer jig and a second outer jig manufactured of a metal material to compress the first and second inner jigs in directions for fixing the customized metal implant, with an insertion recess into which the first and second inner jigs are inserted in close contact with each other being formed in an inner side part of each of the first outer jig and the second outer jig; and
a driving device coupled to the first outer jig and the second outer jig and configured to move the first outer jig or the second outer jig in a direction for compressing the first and second inner jigs,
wherein while the customized metal implant seated in the recessed part is clamped by compression force of the first and second outer jigs compressing the first and second inner jigs, a post-processing of the customized metal implant is capable of being performed.

2. The jig of claim 1, wherein a slide groove is formed in an inner wall surface of each of the first and second outer jigs, and a slide protrusion inserted into and fastened to the slide groove is formed on each of the first and second inner jigs so that the first and second inner jigs are prevented from departing upward from the first and second outer jigs.

3. The jig of claim 1, wherein the driving device comprises:
a support block to which a screw is fastened to be rotatable in place;
a fixed block formed integrally on a first upper surface part of the support block and fastened to the first outer jig;
a moving block disposed movably on a second upper surface part of the support block and fastened to the second outer jig;
a nut block formed on a lower end part of the moving block by protruding therefrom, with the screw being rotatably inserted into and fastened to the nut block; and
a rotating handle mounted on an outer part of the support block and configured to apply rotational force to the screw,
wherein a guide rail is formed on an upper surface part of the support block by protruding therefrom, and a guide groove into which the guide rail is inserted to be fastened thereto is formed in a lower surface part of each of the moving block and the second outer jig.

4. A method for manufacturing a jig for a post-processing of a customized orthopedic implant, the method comprising:
manufacturing a customized metal implant for artificial hip joint surgery by using a 3D printer according to a shape and a damage degree of a patient's hip joint;
manufacturing a first inner jig and a second inner jig made of a polymer resin material by a 3D printer for fixing the customized metal implant for post-processing thereof in such a manner that a recessed part for seating and clamping the customized metal implant is formed in each of the first inner jig and the second inner jig; and
manufacturing a first outer jig and a second outer jig made of a metal material to compress the first and second inner jigs in directions for fixing the customized metal implant in such a manner that an insertion recess into which the first and second inner jigs are inserted in close contact with each other is formed in an inner side part of each of the first outer jig and the second outer jig,
wherein while the customized metal implant seated in the recessed part is clamped by compression force of the first and second outer jigs compressing the first and second inner jigs, the post-processing of the customized metal implant is capable of being performed, and
the first inner jig and the second inner jig are in direct contact with the customized metal implant and are 3D printed with a polyamide material so that the first inner jig and the second inner jig are capable of protecting a surface of the customized metal implant.
